(19) United States
(12) Patent Application Publication
(10) Pub. No.: US 2006/0293736 A1
(43) Pub. Date: Dec. 28, 2006

(54) Compression bandage

(76) Inventors: Louis F. Mariano JR., Hooksett, NH (US); Miles E. Gerrish, Hooksett, NH (US)

Correspondence Address: DEVINE, MILLIMET & BRANCH, P.A., 111 AMHERST STREET, BOX 719, MANCHESTER, NH, 03105, US

(21) Appl. No.: 11/362,246

(22) Filed: Feb. 24, 2006

Related U.S. Application Data
(60) Provisional application No. 60/695,105, filed on Jun. 28, 2005.

(51) Int. Cl. A61F 7/00

(52) U.S. Cl. 607/111; 607/114

(57) ABSTRACT

A compression bandage including a bonded web bandage having a predetermined size and shape and a cooling liquid in contact with the bonded web having an acid pH which is safe for external use on the human body. Also, a method of treating a contusion on a body part. The method includes the following steps. Wrapping a compression bandage around the contused body part whereby the cooling liquid cools the contused body part. The compression bandage includes a bonded web bandage having a predetermined size and shape and a cooling liquid in contact with the bonded web. The cooling liquid has an acid pH which is safe for external use on the human body.

### CROSS-REFERENCE TO RELATED APPLICATIONS

The present application claims the benefit of Provisional Patent Application Ser. No. 60/695,105 filed Jun. 28, 2005, which is incorporated herein by reference.

### TECHNICAL FIELD

The present invention relates to an improved bandage and more particularly, to an improved compression bandage.

### BACKGROUND INFORMATION

There are many bodily injuries, particularly those referred to under the broad designation of contusions. Contusions are painful and cause substantial discomfort to the injured person due to the tissue damage and the resulting hemorrhaging of blood beneath the skin, which in turn causes substantial swelling. Swelling causes the injury to be more painful and disabling. It has long been recognized that, if the swelling can be minimized, then the pain and discomfort can be minimized or eliminated. A contusion, as defined in Taber's Cyclopedic Medical Dictionary, is an injury in which the skin is not broken; a bruise. For this purpose, the conventional treatment of this type of injury has been to apply ice packs or similar devices to the injured portion of the body as soon as possible after the injury in an attempt to minimize the swelling and pain. This technique has limitations and can only be used in specific situations and circumstances. An example of this is when an icepack is applied; the individual has limited mobility and must limit their activities because of the size, weight, and coldness of the icepack applied to the injured area. Thus, the conventional method of treating a contusion is by application of an icepack or other cold apparatus. However, a barrier must also be applied between the icepack and the skin. Thus, mobility is very limited.

Accordingly, there is a need for a bandage that can directly contact the skin, can be quickly and easily wrapped around the injury, allows full mobility, and provides cold compression.

### SUMMARY OF THE INVENTION

In accordance with one aspect of the present invention, a cooling compression bandage. The cooling compression bandage includes a bonded web bandage having a predetermined size and shape and a cooling liquid in contact with the bonded web. The cooling liquid has an acid pH which is safe for external use on the human body.

Some aspects of this embodiment may include: where the bonded web bandage is made from cotton; where the cooling liquid includes ethyl alcohol and water; where the cooling liquid additionally includes at least one selected from this group: anethole NF; anise oil NF; myrcia oil NF; benzaldehyde NF; bergamot oil NF; bitter almond oil NF; camphor USP; cedar leaf USP; chlorothymol NF; cinnamic aldehyde NF; citronella oil natural; clove oil NF; lavender oil NF; methanol USP; methyl salicyclate NF; allyl isothiocyanates UST; peppermint oil NF; phenol USP; phenyl salicylate NF; pine oil NF; pine needle oil dwarf NF; rosemary oil NF; safrole; thyme oil NF; thymol NF; tolu balsam USP; and turpentine oil NF; where the cooling liquid additionally includes camphor crystal and menthol crystal; where the cooling liquid includes ethyl alcohol, camphor crystal, menthol crystal and water.

In accordance with another aspect of the present invention, a method of treating a contusion on a body part. The method includes the following steps. Wrapping a compression bandage around the contused body part whereby the cooling liquid cools the contused body part. The compression bandage includes a bonded web bandage having a predetermined size and shape and a cooling liquid in contact with the bonded web. The cooling liquid has an acid pH which is safe for external use on the human body.

These aspects of the invention are not meant to be exclusive and other features, aspects, and advantages of the present invention will be readily apparent to those of ordinary skill in the art when read in conjunction with the appended claims and accompanying drawings.

### BRIEF DESCRIPTION OF THE DRAWINGS

These and other features and advantages of the present invention will be better understood by reading the following detailed description, taken together with the drawings wherein:

FIG. 1 illustrates one manner of use for the bandage, namely, wrapping of the bandage around an injured hand or wrist;

FIG. 2 illustrates another use of the bandage for wrapping same around an injured ankle or foot;

FIG. 3 is a chart representing results of test subject questionnaires;

FIG. 4 is a chart representing results of test subject questionnaires;

FIG. 5 is a chart representing results of test subject questionnaires;

FIG. 6 is a chart representing results of test subject questionnaires.

### DETAILED DESCRIPTION OF THE PREFERRED EMBODIMENTS

The invention is a compression bandage including a bonded web bandage having a predetermined size and shape and a cooling liquid in contact with the bonded web having an acid pH which is safe for external use on the human body.

The bandage can be any standard bandage, but in the preferred embodiment is a 100% cotton bandage having a standard weave or open weave. The bandage is a bonded web weave, having a standard web weave size. The bandage can be any length and width. Depending on the body area in which it is intended to be wrapped; the bandage width and length will vary.

The bandage can be self-adherent by means of a strip of Velcro™ being sewn onto one end. The bandage is known to those of ordinary skill in the art as a Cotton Crepe Bandage B.P. in the industry. This bandage is readily available throughout the United States. The bandage does not contain any Latex material. The compression is achieved by the weave and natural stretch in the cotton material.

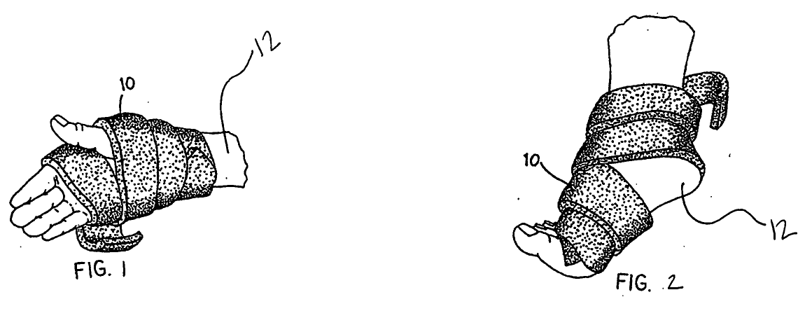

Referring to FIGS. 1 and 2, the preferred embodiment of the present invention is shown. The bandage 10 is applied by wrapping the bandage 10 around the injured area 12 (here, a hand/wrist or foot/ankle). The bandage 10 is wrapped around the injured area 12 using as much compression (the tightness of the wrap) as needed by the injury and slightly overlapping the previous wrap to ensure as much of the bandage as possible is exposed to the air. This ensures even evaporation and provides the maximum cooling effect of the bandage wrap.

The thickness, flexibility and pliability of the bandage are devised so that when the bandage is wrapped around a part of the body, it will conform closely to the contour of the body part in order that the entire inner surface of the strip will be substantially in surface-to-surface contact with the body part. The thickness of the bandage will vary from manufacturer to manufacturer. The width of the bandage is selected so that the body part can be completely wrapped by making only a few turns of the bandage around the body part. The width can be from 2 inches to 12 inches. The length of the bandage is selected such that it is effective to wrap the entire area. For example, the length of the bandage can be from approximately 1 to approximately 12 feet.

A cooling liquid is in contact with the bandage. The bandage therefore has the cooling liquid contained and the invention is completely self-contained. In the preferred embodiment, the cooling liquid is an Ethyl Alcohol, Camphor Crystal, Menthol Crystal and water solution. This solution is known in the industry as SDA38B (Special Denatured Alcohol Formula 38B) an industry standard formula. In the preferred embodiment, the SDA38B formula is diluted such that the solution is 3 parts water and 1 part SDA38B Formula. In other embodiments, the SDA38B formula ethanol is diluted such that the solution is 4 parts water and 1 part SDA38B formula ethanol. In other embodiments, the cooling liquid contains ethyl alcohol and water.

In the preferred embodiment, a bandage having dimensions of 3 inches wide×4 feet long contains 1 ounce or 30 cc-diluted solution. In the preferred embodiment, a bandage having dimensions of 4 inches wide×4 feet long contains 1 ounce or 30 cc-diluted solution. In the preferred embodiment, a bandage having dimensions of 6 inches wide×4 feet long contains 1 ounce or 30 cc-diluted solution. In the preferred embodiment, a bandage having dimensions of 4 inches wide×12 feet long contains 3 ounces or 90 cc-diluted solution. In the preferred embodiment, a bandage having dimensions of 6 inches wide×12 feet long contains 4 ounces or 120 cc-diluted solution. In other embodiments, bandages having differed dimensions include an equal amount of solution per square area of bandage.

In alternate embodiments, in addition to ethanol and water, the solution can include at least a trace amount of one or more of the following: Anethole NF, Anise oil NF, Bay oil (Myrcia Oil) NF, Benzaldehyde NF, Bergamot Oil NF, Bitter Almond Oil NF, Camphor USP, Cedar Leaf USP, Chlorothymol NF, Cinnamic Aldehyde NF, Cinnamon Oil NF, Citronella Oil Natural, Clove Oil NF, Coal Tarr USP, Eucalyptol NF, Eucalyptus Oil NF, Eugenol USP, Gualocol NF, Lavender Oil NF, Methanol USP, Methyl Salicyclate NF, Mustard Oil Volatile (Allyl Isothiocyanates) UST, Peppermint Oil NF, Phenol USP, Phenyl Salicylate (Salol) NF, Pine Oil NF, Pine Needle Oil Dwarf NF, Rosemary Oil NF, Safrole, Thyme Oil NF, Thymol NF, Tolu Balsam USP, Turpentine Oil NF. Additionally, the solution can contain any other fragrance known by one of ordinary skill in the art. The solution can contain any compound having effects on the skin, for example, moisturizers. The solution can contain a dye or coloring agent.

The product can be manufactured by hand by putting a rolled bandage into an empty package and adding the correct amount of fluid based on the size of the bandage, and then the package can be hand sealed with a heat sealer.

In the preferred embodiment, the package is made from any water tight material and, when sealed, is air tight. The package contains the bandage and cooling liquid. As discussed above, the amount of cooling liquid or fluid varies depending on the size of the bandage.

The manufacturing process can also be automated to increase volume and bring the manufacturing cost down by using an automated packaging machine that will put a rolled bandage into a package, fill with the correct amount of fluid (the machine is preset by bandage size), and seal automatically. Some machines will produce up to 1000 units per hour.

The cooling effect will last up to two hours, no matter what size.

### EXAMPLES

Test Subjects

Testing was done using 50 test subjects. The test subjects used were United States Marines, both men and women ages 18-29. Each test subject was suffering from a field injury, including twisted knee, sprained ankle, heat related injury, sore muscles, and sore joints.

Cooling Compression bandage

A cooling compression bandage according to the present specification was used. The bandage used was approximately 4 inches wide by 48 inches stretched. The cooling liquid used was according to the specification.

Test Environment

All testing was conducted in a battlefield environment for both day and night operations.

Application

The bandage was applied to the test subjects at the point of injury. The bandage provided cooling to the injured area for 2 hours. Following, the bandage was left on the body to provide support to the injured area.

Questionnaire

Following testing, each test subject completed a questionnaire.

Summary of Results

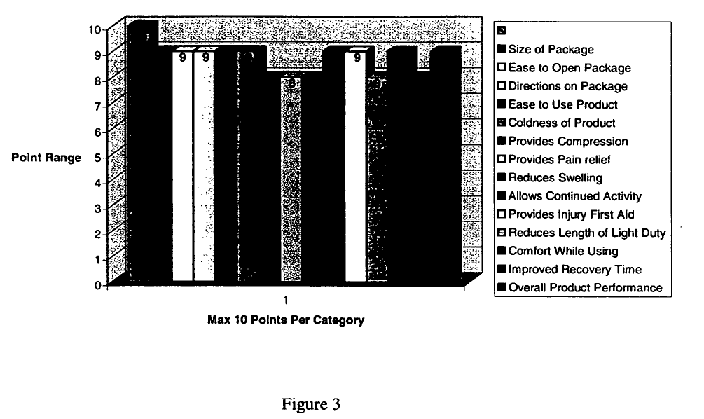

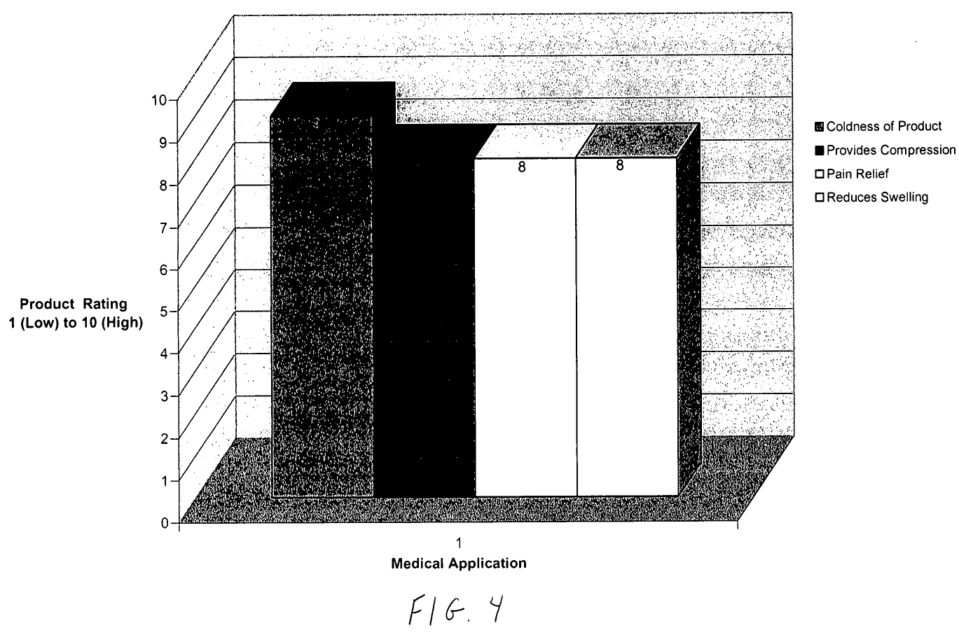

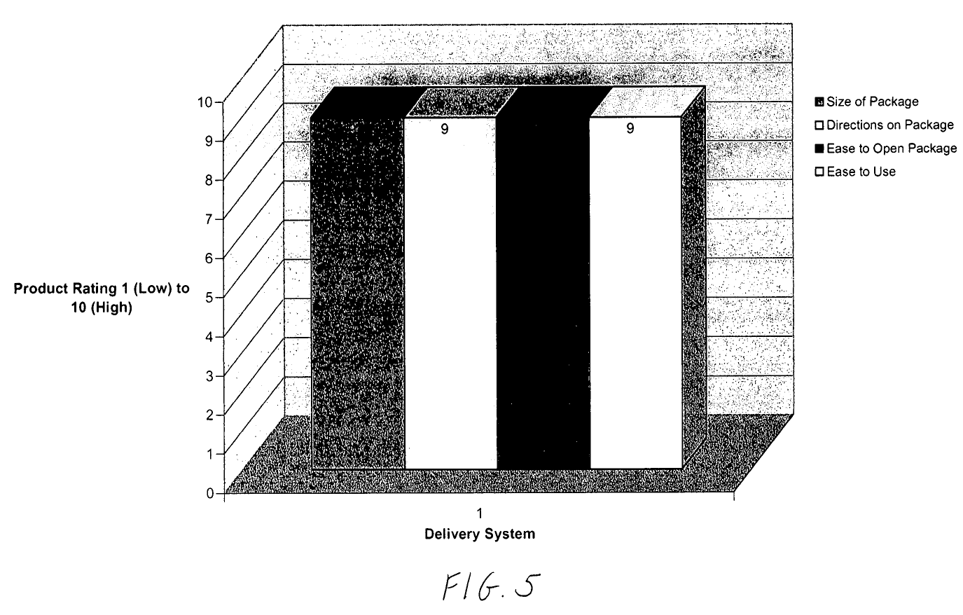

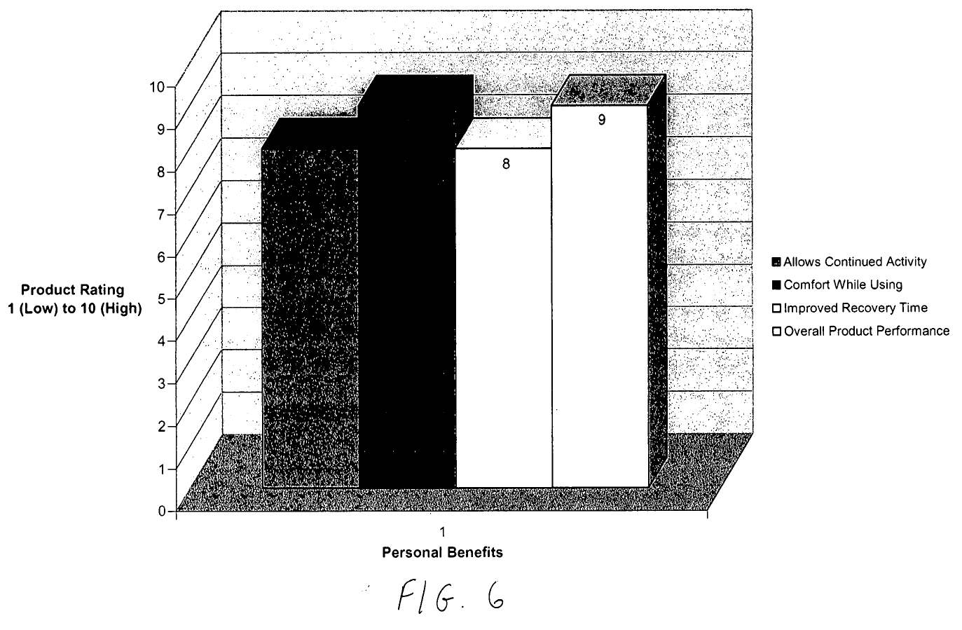

The questionnaire results are represented in FIGS. 3-6. While FIG. 3 represents a summary of all the different categories. FIGS. 4-6 each represent only specific categories. The test subjects rates the compression bandage on a scale from 1-10, 10 being the highest. The categories were as follows: size of package; directions on package; east to open package; ease of use; coldness of product; provides compression; pain relief; reduces swelling; allows continued activity; comfort while using; improved recovery time; and overall product performance.

While the principles of the invention have been described herein, it is to be understood by those skilled in the art that this description is made only by way of example and not as a limitation as to the scope of the invention. Other embodiments are contemplated within the scope of the present invention in addition to the exemplary embodiments shown and described herein. Modifications and substitutions by one of ordinary skill in the art are considered to be within the scope of the present invention.

1. A cooling compression bandage comprising:
a bonded web bandage having a predetermined size and shape; and
a cooling liquid in contact with said bonded web having an acid pH which is safe for external use on the human body.

2. The cooling compression bandage of claim 1 wherein said bonded web bandage is made from cotton.

3. The cooling compression bandage of claim 1 wherein said cooling liquid comprising:
ethyl alcohol; and
water.

4. The cooling liquid of claim 3 further comprising at least one selected from the group consisting of: anethole NF; anise oil NF; myrcia oil NF; benzaldehyde NF; bergamot oil NF; bitter almond oil NF; camphor USP; cedar leaf USP; chlorothymol NF; cinnamic aldehyde NF; citronella oil natural; clove oil NF; lavender oil NF; methanol USP; methyl salicyclate NF; allyl isothiocyanates UST; peppermint oil NF; phenol USP; phenyl salicylate NF; pine oil NF; pine needle oil dwarf NF; rosemary oil NF; safrole; thyme oil NF; thymol NF; tolu balsam USP; and turpentine oil NF.

5. The cooling liquid of claim 3 further comprising:
camphor crystal; and
menthol crystal.

6. The cooling compression bandage of claim 1 wherein said cooling liquid comprising:
ethyl alcohol;
camphor crystal;
menthol crystal; and
water.

7. The cooling liquid of claim 6 further comprising at least one selected from the group consisting of: anethole NF; anise oil NF; myrcia oil NF; benzaldehyde NF; bergamot oil NF; bitter almond oil NF; camphor USP; cedar leaf USP; chlorothymol NF; cinnamic aldehyde NF; citronella oil natural; clove oil NF; lavender oil NF; methanol USP; methyl salicyclate NF; allyl isothiocyanates UST; peppermint oil NF; phenol USP; phenyl salicylate NF; pine oil NF; pine needle oil dwarf NF; rosemary oil NF; safrole; thyme oil NF; thymol NF; tolu balsam USP; and turpentine oil NF.

8. A method of treating a contusion on a body part, said method comprising the steps of:
wrapping a compression bandage as claimed in claim 1 around the contused body part whereby said cooling liquid cools said contused body part.
